(19) United States
(12) Patent Application Publication
(10) Pub. No.: US 2011/0257582 A1
(43) Pub. Date: Oct. 20, 2011

(54) MEDICATED SHEET

(76) Inventors: Wataru WATANABE, Usuki (JP); Yuichiro Asano, Tokyo (JP); Yozo Niina, Usuki (JP)

(21) Appl. No.: 12/868,789

(22) Filed: Aug. 26, 2010

(30) Foreign Application Priority Data
Apr. 20, 2010 (JP) 2010-096841

(51) Int. Cl. A61N 1/30

(52) U.S. Cl. 604/20

(57) ABSTRACT

An object of the present invention is to safely discard the electrode plate having pin holder-shaped projections of a medicated sheet by incineration. An electrode plate with pin holder-shaped projections is made from a conductive resin in which carbon black serving as a conductive substance is mixed to a thermoplastic resin, so that the electrode plate including the pin holder-shaped projections can be safely discarded by incineration.

### BACKGROUND OF THE INVENTION

(1) Field of the Invention

The present invention relates to a wet medicated sheet in which medicinal agents containing electrolytic or non-electrolytic pharmaceutical ingredients are impregnated or applied on the medication surface to be placed on the skin.

(2) Description of the Related Art

The wet medicated sheet in which the medicinal agent is impregnated or applied on the medication surface to be placed on the skin so that the pharmaceutical ingredients can be absorbed into the body from the skin is widely used to treat nerve pain, shoulder stiffness, and the like as easy and convenient treatment means. However, in the conventional medicated sheet, only about 20 to 30% of the pharmaceutical ingredients are absorbed from the skin at the most, and thus the absorption efficiency of the pharmaceutical ingredients is known to be low.

One of the inventors of the present invention first developed, as an electrical treatment equipment using ion infiltration effect of the pharmaceutical ingredients by current flowing through the skin, an equipment in which an outer surface of an electrode on one surface of a flat compact battery and a peripheral side surface of the battery are covered with an external capsule material by way of a desired gap, the relevant electrode and another electrode are insulated with an insulating material, a medicinal agent having fluidity added with conductivity is filled to the gap, the open portion at the peripheral side of the gap is sealed with a strippable sheet, and the other electrode surface stripped with the sheet is contacted to the skin in time of use (see Japanese Patent Publication No. 3-69545), and then developed, as an electrical treatment equipment that can be used by simply being attached to the medicated sheet in which the medicinal agent containing electrolytic pharmaceutical ingredients, which ionize, is impregnated or applied on the medication surface, and that enhances the absorption efficiency of the pharmaceutical ingredients of the medicated sheet, an electrical treatment equipment in which a battery is formed with an electrolytic solution interposed between a positive electrode plate to be attached to the medication surface of the medicated sheet and a negative electrode plate to be contacted to the skin, and the outer peripheral part between the electrode plates is sealed with an annular seal member having insulating property (see Japanese Laid-open Patent Publication No. 2005-192848).

Recently, it is reported that the absorption of the pharmaceutical ingredients can be accelerated by the convection flow of water that occurs by flowing current to the skin through the use of the electrical treatment equipment as described in Japanese Laid-open Patent Publication No. 2005-192848 even with the medicated sheet that uses that medicinal agent containing non-electrolytic pharmaceutical ingredients dissolved or dispersed in a solvent (see Seiji, Higo “Regarding recent development trend of Transdermal Drug Delivery System”, YAKUGAKU ZASSHI, The Pharmaceutical Society of Japan, 2007, Vol. 127, No. 4, p. 655-662).

To further enhance the absorption efficiency of the electrolytic and non-electrolytic pharmaceutical ingredients, the inventors of the present invention developed an electrical treatment equipment and a medicated sheet in which a microscopic pin holder-shaped projection is arranged on the skin contacting surface of the electrode plate, and the pharmaceutical ingredients are injected along the pin holder-shaped projection to be inserted under the skin (see Japanese Patent Publication No. 4170307, Japanese Laid-open Patent Publication No. 2009-136383, and Japanese Laid-open Patent Publication No. 2009-142432). The electrical treatment equipment described in Japanese Patent Publication No. 4170307 includes the pin holder-shaped projection on the skin contacting surface of the positive electrode plate made from stainless steel or the negative electrode plate made from titanium. Furthermore, the medicated sheet described in Japanese Laid-open Patent Publication No. 2009-136383 and Japanese Laid-open Patent Publication No. 2009-142432 has a pair of electrode plates, made from two types of metals having different normal electrode potential difference or subjected to metal plating, attached to the medication surface impregnated or applied with medicinal agent so as to be insulated from each other, where the pin holder-shaped projection is arranged on the skin contacting surface of one or both electrode plates.

### SUMMARY OF THE INVENTION

The electrical treatment equipment described in Japanese Patent Publication No. 4170307 and the medicated sheet described in Japanese Laid-open Patent Publication No. 2009-136383 and Japanese Laid-open Patent Publication No. 2009-142432 have a microscopic pin holder-shaped projection arranged on the skin contacting surface of the electrode plate, and thus can significantly enhance the absorption efficiency of the pharmaceutical ingredients by the injection effect to under the skin by the pin holder-shaped projection, and medicinal agents such as anesthetic drugs and vaccine that are conventionally injection-administered using a syringe can be injection-administered other than medicinal agents of anti-inflammatory analgesic plaster. However, the electrode plate with the pointed pin holder-shaped projection like the syringe needle becomes a dangerous medical waste after use, and the problem arises in that it cannot be safely discarded by incineration since the electrode plate that becomes the dangerous medical waste is made from metal or is subjected to metal plating.

It is an object of the present invention to safely discard the electrode plate with a pin holder-shaped projection of the medicated sheet by incineration.

To solve the above problem, the present invention adopted a configuration of a medicated sheet in which positive and negative electrode plates, which are insulated from each other, are arranged on a medication surface of a medicated cloth impregnated or applied with medicinal agent, and microscopic pin holder-shaped projections are arranged on a skin contacting surface of at least one electrode plate to be contacted to the skin, wherein the electrode plate including the pin holder-shaped projections is made from a conductive resin.

In other words, the electrode plate including the pin holder-shaped projections is safely discarded by incineration by forming the electrode plate including the pin holder-shaped projections from a conductive resin. The pharmaceutical ingredients of the medicinal agent may positively ionize, negatively ionize, or may not ionize, where the electrode plate that contacts the skin is preferably negative if the pharmaceutical ingredients positively ionizes and the electrode plate that contacts the skin is preferably positive if the pharmaceutical ingredients negatively ionizes.

The conductive resin can be obtained by mixing a conductive substance to a thermoplastic resin.

The incineration efficiency can be enhanced if the conductive substance is carbon black.

The pin holder-shaped projections of the electrode plate made from the conductive resin are made through an injection-molding method, so that the electrode plate including the pin holder-shaped projections can be efficiently and inexpensively manufactured.

The height of the pin holder-shaped projection is preferably between 50 and 600 μm. The skin is made up of a keratinous layer, an epidermis including living cells, and a dermis where capillary blood vessels and nerves are present from the surface side, where the height of the projection is preferably about 50 to 150 μm when injecting the pharmaceutical ingredients of an anti-inflammatory analgesic plaster, and the like to the epidermis, and the height of the projection is preferably high or about 150 to 600 μm when injection-administering the pharmaceutical ingredients such as vaccine to the dermis. In the case of injection-administering the pharmaceutical ingredients to the dermis, the medicated sheet is made disposable so as to eliminate the possibility of bacterial infection, and the like from the capillary blood vessel of the dermis.

One of the positive or negative electrode plates is made from a conductive resin including the pin holder-shaped projections, and the other electrode plate is made from metal having a normal electrode potential difference with the one electrode plate made from the conductive resin or is subjected to metal plating, or is made from a conductive resin mixed with a conductive substance having a normal electrode potential difference with the one electrode plate, so that current that passes through the skin and the medicinal agent of the medication surface is generated between the electrode plates by the potential difference between the positive and negative electrode plates, a separate battery becomes unnecessary, and incineration can be facilitated. If the other electrode plate is also made from a conductive resin mixed with the conductive substance, the entire medicated sheet can be incinerated as a whole.

If the conductive substance to be mixed to the conductive resin is carbon black, the normal electrode potential becomes high or the same extent as the noble platinum. If the conductive substance is a metal filler, or the like, it becomes the normal electrode potential of the relevant metal. Therefore, the conductive resin that uses carbon black for the conductive substance is preferably for the positive electrode plate. In this case, the negative electrode plate may be made from a base metal such as zinc and magnesium having low normal electrode potential or may be subjected to plating of such base metal, or may be made from a conductive resin mixed with base metal filler, and the like.

An overlapping margin portion is arranged at the positive and negative electrode plates, and an electrolytic solution is interposed between the positive and negative electrode plates at the overlapping margin portion, so that the battery is formed at the overlapping margin portion of the positive and negative electrode plates and the current that passes through the skin can be actively generated.

The current that passes through the skin can be actively generated even by applying voltage from an external power supply to between the positive and negative electrode plates.

The entire medicated sheet can be incinerated as a whole by forming both the positive and negative electrode plates from a conductive resin.

The medicated sheet of the present invention has the electrode plate including the pin holder-shaped projections arranged on the medication surface of the medicated cloth made from a conductive resin, and hence the electrode plate including the pin holder-shaped projections can be safely discarded by incineration.

### BRIEF DESCRIPTION OF THE DRAWINGS

FIG. 1 is a longitudinal side view showing a medicated sheet of a first embodiment;

FIG. 2 is a traverse plan view taken along a line II-II in FIG. 1;

FIG. 3 is a longitudinal side view showing an overlapping margin portion of the positive and negative plates in FIG. 1 in an enlarged manner;

FIG. 4 is a longitudinal side view showing a medicated sheet of a second embodiment;

FIG. 5 is a traverse plan view taken along a line V-V in FIG. 4;

FIG. 6 is a longitudinal side view showing an overlapping margin portion of the positive and negative plates in FIG. 4 in an enlarged manner;

FIG. 7 is a longitudinal side view showing a medicated sheet of a third embodiment;

FIG. 8 is a traverse plan view taken along a line VIII-VIII in FIG. 7;

FIG. 9 is a longitudinal side view showing a medicated sheet of a fourth embodiment; and

FIG. 10 is a traverse plan view taken along a line X-X in FIG. 9.

### DETAILED DESCRIPTION OF THE PREFERRED EMBODIMENTS

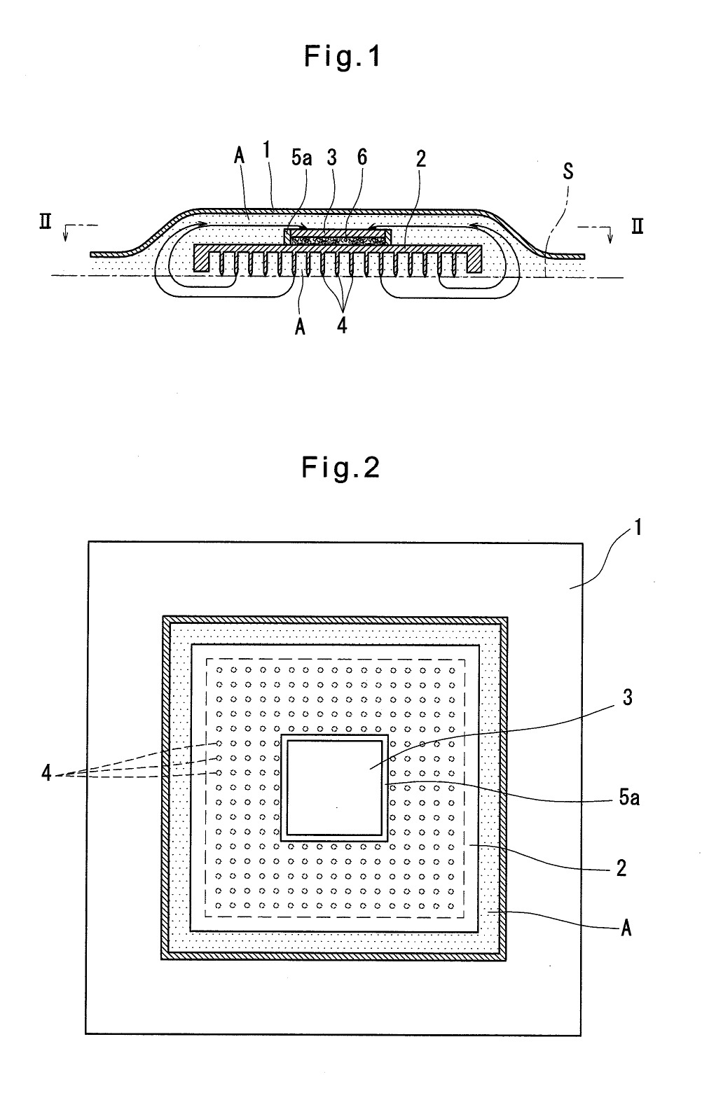

Embodiments of the present invention will be described based on the drawings. FIGS. 1 to 3 show a first embodiment. As shown in FIGS. 1 and 2, the medicated sheet has a positive electrode plate 2 having a large diameter made from a conductive resin, in which carbon black having high normal electrode potential is mixed to thermoplastic resin, and a negative electrode plate 3 having a small diameter made from a conductive resin, in which zinc filler having low normal electrode potential is mixed to thermoplastic resin, arranged to include an overlapping margin portion on a medication surface of a medicated cloth 1 applied with a medicinal agent A containing electrolytic pharmaceutical ingredients that negatively ionize, and the positive electrode plate 2 is arranged to face the skin S. The planar shapes of the medicated cloth 1 and the positive and negative electrode plates 2, 3 are a square concentric with respect to each other, but the planar shapes may be an arbitrary shape.

Microscopic pin holder-shaped projections 4 are arranged over the entire surface on a skin contacting surface of the positive electrode plate 2, and the medicinal agent A is also filled between the pin holder-shaped projections 4. The pin holder-shaped projection 4 has a height of between 50 and 600 μm, and is formed by injection-molding the conductive resin. In the following drawings starting with FIG. 1, the height and the interval of the projections 4 are illustrated to be greater than the actual dimension to facilitate the understanding of the projection 4. Therefore, the electrode plate including the microscopic pin holder-shaped projections 4 can be efficiently and inexpensively manufactured. Furthermore, the medicated sheet can be entirely incinerated as a whole including the positive electrode plate 2 with the pin holder-shaped projections 4 since the positive and negative electrode plates 2, 3 are made from conductive resin.

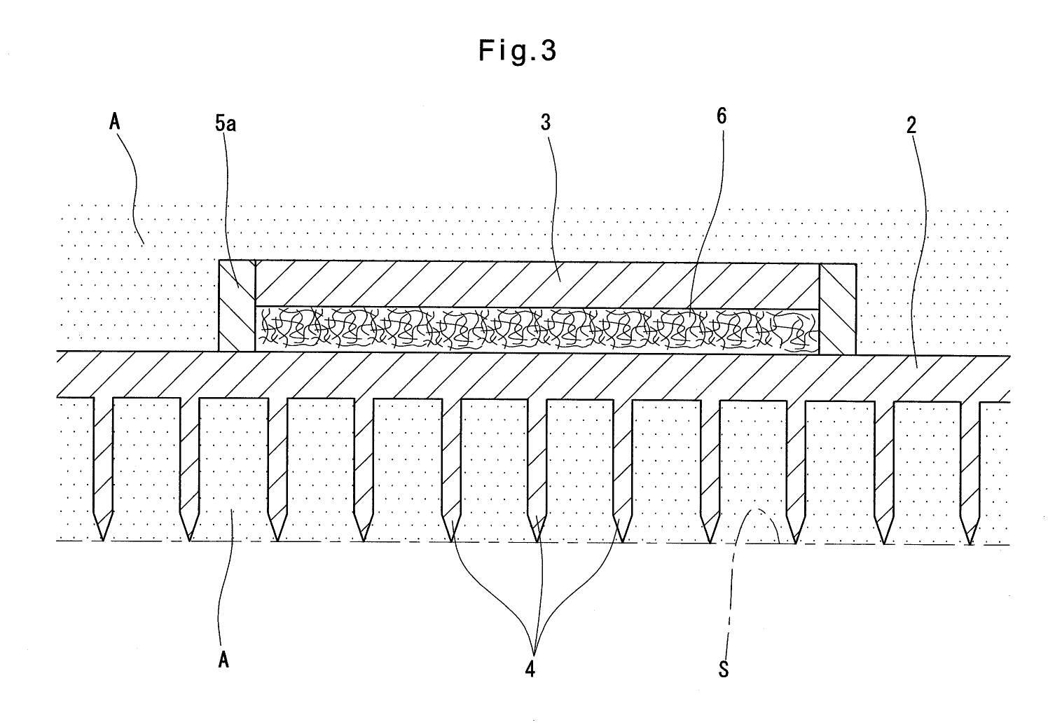

As shown in an enlarged manner in FIG. 3, a square ring-shaped insulating member 5a made from resin is attached to the periphery of the overlapping margin portion of the positive and negative electrode plates 2, 3 so as to insulate the positive and negative electrode plates 2, 3, and a separator 6 in which electrolytic solution is impregnated to a non-woven cloth is interposed between the positive and negative electrode plates 2, 3 of the overlapping margin portion, thereby forming a battery at the overlapping margin portion of the positive and negative electrode plates 2, 3. Therefore, as shown with an arrow in FIG. 1, the current flows through the skin S and the medicinal agent A of the medication surface from the positive electrode plate 2 of high normal electrode potential to the negative electrode plate 3 of low normal electrode potential by the electromotive force of the battery, and the pharmaceutical ingredients that negatively ionize in the medicinal agent A moves from the medication surface towards the skin S in a direction opposite to the flow of the current, and is absorbed into the body from the skin S and the medicinal agent A filled between the pin holder-shaped projections 4 is injected to under the skin.

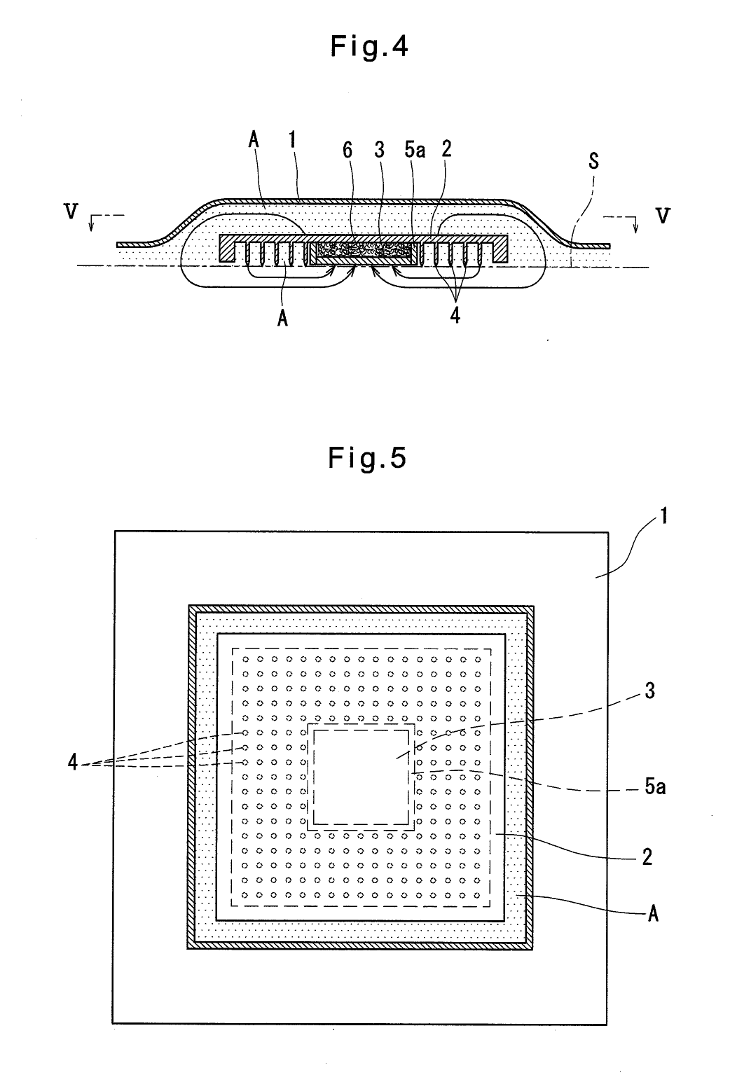

FIGS. 4 to 6 show a second embodiment. This medicated sheet has the medicinal agent A containing the electrolytic pharmaceutical ingredients, which positively ionize, applied to the medication surface of the medicated cloth 1, where the negative electrode plate 3 is overlapped on the skin contacting surface side of the positive electrode plate 2 so that the negative electrode plate 3 contacts the skin S, the microscopic pin holder-shaped projections 4 are arranged on the skin contacting surface of the positive electrode plate 2 excluding the overlapping margin portion with the negative electrode plate 3, and the medicinal agent A is also filled between the projections 4, as shown in FIGS. 4 and 5. Other portions are the same as the first embodiment, and the positive electrode plate 2 is made from a conductive resin mixed with carbon black, and the negative electrode plate 3 is made from a conductive resin mixed with zinc filler. Therefore, this medicated sheet also can be entirely incinerated as a whole since the positive and negative electrode plates 2, 3 are made from conductive resin.

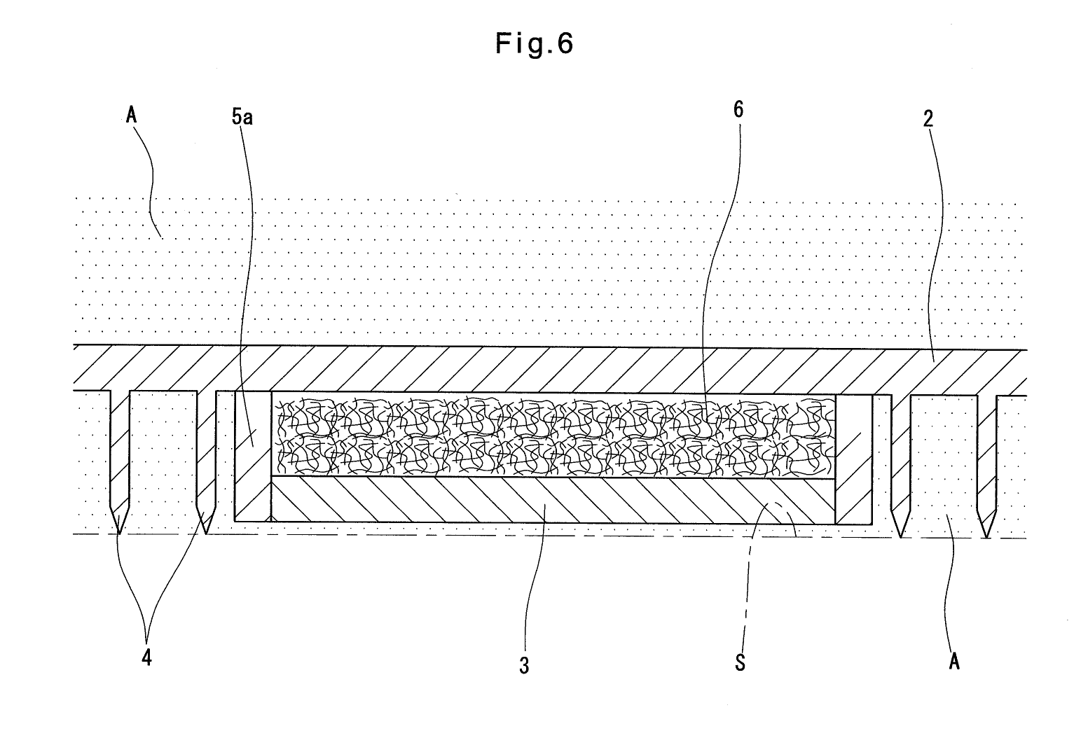

As shown in an enlarged manner in FIG. 6, in the present embodiment as well, the periphery of the overlapping margin portion of the positive and negative electrode plates 2, 3 is insulated by the square ring-shaped insulating member 5a, and the separator 6 in which electrolytic solution is impregnated to a non-woven cloth is interposed between the positive and negative electrode plates 2, 3, thereby forming a battery at the overlapping margin portion of the positive and negative electrode plates 2, 3. Therefore, as shown with an arrow in FIG. 4, the current flows through the skin S and the medicinal agent A of the medication surface from the positive electrode plate 2 of high normal electrode potential to the negative electrode plate 3 of low normal electrode potential by the electromotive force of the battery, and the pharmaceutical ingredients that positively ionize in the medicinal agent A moves from the medication surface towards the skin S in the direction same as the flow of the current, and is absorbed into the body from the skin S and the medicinal agent A filled between the pin holder-shaped projections 4 is injected to under the skin.

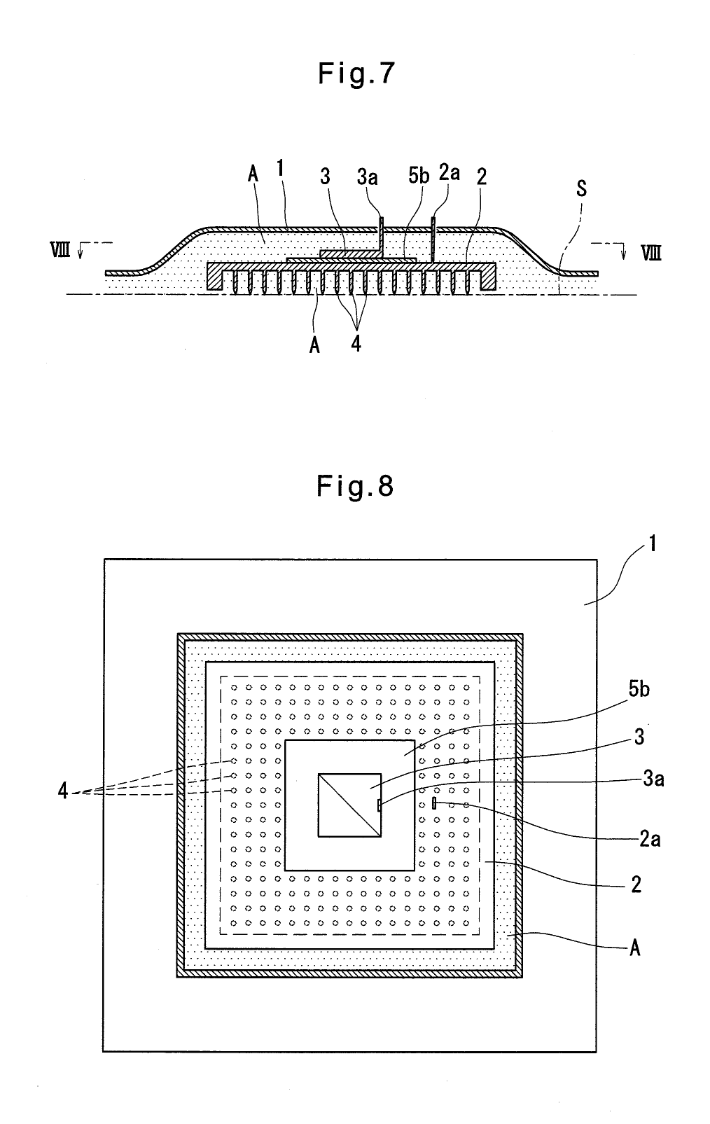

FIGS. 7 and 8 show a third embodiment. The medicated sheet has the two electrode plates 2, 3, one of which becomes positive or negative, made from a conductive resin mixed with carbon black, where the electrode plate 3 having a small diameter is overlapped, so as to include an overlapping margin portion, on the medication surface side of the electrode plate 2 having a large diameter in which the microscopic pin holder-shaped projections 4 are arranged on the skin contacting surface, the electrode plates 2, 3 are insulated by an insulating member 5b made from a resin film, lead portion 2a, 3a is arranged at each electrode plate 2, 3, and the lead portions 2a, 3a are exposed to the outer side of the medicated cloth 1 so as to be connected to an external power supply (not shown). Each lead portion 2a, 3a may be obtained by wire-connecting a conductive wire, and the like to each electrode plate 2, 3. Other portions are the same as the first embodiment, and the pin holder-shaped projections 4 are arranged over the entire skin contacting surface of the electrode plate 2, and the medicinal agent A is filled between the projections 4.

Therefore, in the present embodiment, the positive and negative poles of the two electrode plates 2, 3 can be arbitrarily changed by connecting the external power supply to the lead portion 2a, 3a of each electrode plate 2, 3 so that the current flows through the skin S and the medicinal agent A of the medication surface from the electrode plate that becomes positive to the electrode plate that becomes negative and response can be made even when the pharmaceutical ingredients positively ionize or negatively ionize, and the voltage between the electrode plates 2, 3 can be arbitrarily set according to the degree of necessity of treatment. The voltage that fluctuates to a pulse form and the like can also be applied. Furthermore, this medicated sheet can also be entirely incinerated as a whole.

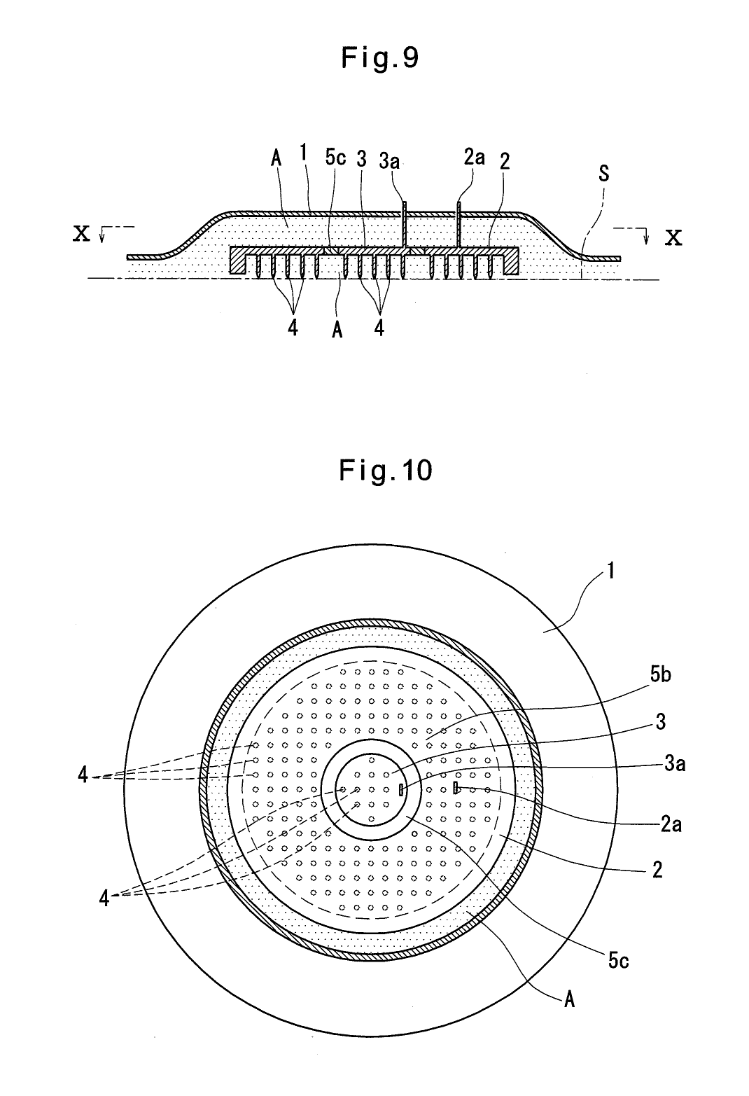

FIGS. 9 and 10 show a fourth embodiment. This medicated sheet also has the two electrode plates 2, 3, one of which becomes positive or negative, made from a conductive resin mixed with carbon black. In the present embodiment, the circular plate-shaped electrode plate 3 having a small diameter is arranged in the same plane on the inner side of the circular ring-shaped electrode plate 2 having a large diameter, the microscopic pin holder-shaped projections 4 are arranged on the skin contacting surface of both electrode plates 2, 3, and the medicinal agent A is filled between the projections 4. The electrode plates 2, 3 are insulated with an annular insulating member 5c, and the lead portion 2a, 3a to be exposed to the outer side of the medicated cloth 1 is arranged on each electrode plate 2, 3. Therefore, this medicated sheet can also arbitrarily change the positive state and the negative state of the two electrode plates 2, 3 by connecting the external power supply to each lead portion 2a, 3a, and can also arbitrarily set the voltage between the electrode plates 2, 3. The medicated sheet can also be entirely incinerated as a whole.

In each embodiment described above, both electrode plates are made from a conductive resin, but the electrode plate not arranged with the pin holder-shaped projection made be made from metal or may be subjected to metal plating.

In each embodiment described above, the medicinal agent is filled between the pin holder-shaped projections arranged on the skin contacting surface of the electrode plate, but a small hole, a slit, or the like may be formed in the electrode plate including the pin holder-shaped projections, so that the medicinal agent applied or impregnated to the medication surface exudes out to the skin contacting surface side from the small hole, the slit, or the like.

In each embodiment described above, the pharmaceutical ingredients of the medicinal agent are electrolytic, but the medicated sheet according to the present invention may be applied to that in which the pharmaceutical ingredients are non-electrolytic.

What is claimed is:

1. A medicated sheet in which positive and negative electrode plates, which are insulated from each other, are arranged on a medication surface of a medicated cloth impregnated or applied with a medicinal agent, and microscopic pin holder-shaped projections are arranged on a skin contacting surface of at least one electrode plate to be contacted to the skin, wherein the electrode plate including the pin holder-shaped projections is made from a conductive resin.

2. The medicated sheet according to claim 1, wherein the conductive resin is obtained by mixing a conductive substance to a thermoplastic resin.

3. The medicated sheet according to claim 2, wherein the conductive substance is carbon black.

4. The medicated sheet according to claim 2, wherein the pin holder-shaped projections of the electrode plate made from the conductive resin are made through an injection-molding method.

5. The medicated sheet according to claim 1, wherein height of the pin holder-shaped projection is between 50 and 600 μm.

6. The medicated sheet according to claim 1, wherein one of the positive and negative electrode plates is made from a conductive resin including the pin holder-shaped projections, and the other electrode plate is made from metal having a normal electrode potential difference with the one electrode plate made from the conductive resin or is subjected to metal plating, or is made from a conductive resin mixed with a conductive substance having a normal electrode potential difference with the one electrode plate.

7. The medicated sheet according to claim 6, wherein an overlapping margin portion is arranged at the positive and negative electrode plates, and an electrolytic solution is interposed between the positive and negative electrode plates at the overlapping margin portion.

8. The medicated sheet according to claim 1, wherein voltage is applied from an external power supply to between the positive and negative electrode plates.

9. The medicated sheet according to claim 8, wherein both the positive and negative electrode plates are made from a conductive resin.
